(19) United States
(12) Patent Application Publication
(10) Pub. No.: US 2005/0240284 A1
(43) Pub. Date: Oct. 27, 2005

(54) Prosthetic foot devices

(76) Inventor: Daniel Andrew Allert, Austin, TX (US)

Correspondence Address: MICHAEL O. SCHEINBERG, P.O. BOX 164140, AUSTIN, TX, 78716-4140, US

(21) Appl. No.: 10/832,610

(22) Filed: Apr. 27, 2004

(51) Int. Cl. A61F 2/66

(52) U.S. Cl. 623/55

(57) ABSTRACT

With the present invention, improved prosthetic foot devices including whole foot devices, heel assembly devices and forefoot devices are provided that address these and other concerns. For example, a prosthetic foot device is provided that includes a heel assembly coupled to a forefoot device through a rotary flexure coupling. In one embodiment, a heel assembly is provided that comprises a resilient heel member and a heel mount. The heel mount is adapted for connection in the prosthetic foot. It has a contact surface for engaging a portion of the heel member to establish in it an effective spring length. The contact surface engages different portions of the heel member for different phases of a gait cycle when the heel member is being loaded thereby effectively shortening the heel member's spring length and providing it with a non-linear loading response as it is being depressed in the gait cycle. In another embodiment, a forefoot is provided that includes a proximal end and a distal end. The proximal end is adapted to be mounted in the prosthetic foot, e.g., to a distal end of a rotary flexure coupling device. The distal end is concavely curved towards a user's limb. The distal end has a relatively longer and less resilient inner forefoot portion and a relatively shorter and more resilient outer forefoot portion. The inner forefoot portion having a relatively forward weakened flexure region, and the outer forefoot portion has a relatively rearward weakened flexure region. In use, the mean line of flexure of the forefoot portions is between the outer and inner flexure regions and substantially parallel to, and forwardly displaced from, the Tc axis of rotation of an equivalent intact foot.

### TECHNICAL FIELD OF THE INVENTION

The present invention relates to a prosthetic foot. More particularly, it relates to an improved prosthetic foot with characteristics of a dynamic response device.

### BACKGROUND OF THE INVENTION

Prosthetic feet have undergone major developments in the past several decades, largely spurred by patients demanding full functionality in their prosthesis. Bioengineering research has begun to consider the presence of many complex inter-functionalities in the human form and to address these with a more sophisticated prosthetic design.

There are two general types of current, high-end prosthetic feet: dynamic response and articulating. Dynamic response feet are feet that may be semi-rigid or have a flexible keel, while articulating devices attempt to recreate foot and ankle function.

Popular articulating type prosthetic designs include the Navy ankle, the Greissinger foot, the SACH (Solid Ankle Cushioned Heel) foot, and the Tru-Step™ foot, all of which employ rubber spacers to allow flexure and impact absorption. The benefits of these feet are many, including the fact that they generally have good re-creation of the foot's intact functioning. Unfortunately, their extremely high maintenance and material fatigue make them less than optimal. In addition, they have other drawbacks, including their relatively high weight, complexity of construction, noise resulting from pivoting at bushings, high maintenance schedule, and threat of catastrophic failure.

On the other hand, dynamic devices are typically lightweight and relatively highly stable. A popular, exemplary conventional dynamic response type foot is known as the Flex-Foot™. It incorporates a flexible carbon fiber shank and heel spring that allows the entire length of the prosthesis (rather than just the foot) to flex, absorb and return energy. Other dynamic response prosthetic feet are currently available with a range of different approaches. Generally using some type of composite (laminated or injection moulded) in conjunction with metallic hardware, they are conjoined to an endo-skeletal assembly, which joins the prosthetic foot to the stump socket of the wearer. The carbon beam types are quite popular with users because of their robust and lightweight nature. In some models, the laminated beams may be split down center-line, allowing for either side of the foot to move relatively independently of the other, providing increased response and stability. Unfortunately, however, the presently available dynamic response type devices lack the flexibility and accurate ankle replication response of the articulating devices. In addition, many of the dynamic devices require a dedicated type of leg shaft. Accordingly, what is need is an improved prosthetic foot design having benefits of both dynamic response and articulating designs.

### SUMMARY OF THE INVENTION

With the present invention, improved prosthetic foot devices, including whole foot devices, heel assembly devices and forefoot devices, are provided that address these and other concerns. For example, a prosthetic foot device is provided that includes a heel assembly coupled to a forefoot device through a rotary flexure coupling. In one embodiment, a heel assembly is provided that comprises a resilient heel member and a heel mount. The heel mount is adapted for connection in the prosthetic foot. It has a contact surface for engaging a portion of the heel member to establish in it an effective spring length. The contact surface engages different portions of the heel member for different phases of a gait cycle when the heel member is being loaded, thereby effectively shortening the heel member's spring length and providing it with a non-linear loading response as it is being depressed in the gait cycle.

In another embodiment, a forefoot is provided that includes a proximal end and a distal end. The proximal end is adapted to be mounted in the prosthetic foot, e.g., to a distal end of a rotary flexure coupling device. The distal end is concavely curved towards a user's limb. The distal end has a relatively longer and less resilient inner forefoot portion and a relatively shorter and more resilient outer forefoot portion. The inner forefoot portion having a relatively forward weakened flexure region, and the outer forefoot portion having a relatively rearward, weakened flexure region. In use, the mean line of flexure of the forefoot portions is between the outer and inner flexure regions and substantially parallel to, and forwardly displaced from, the T_{c} axis of rotation of an equivalent intact foot.

The foregoing has outlined rather broadly some of the features and technical advantages of the present invention in order that the detailed description of the invention that follows may be better understood. Additional features and advantages of the invention will be described hereinafter. It should be appreciated by those skilled in the art that the conception and specific embodiment disclosed may be readily utilized as a basis for modifying or designing other structures for carrying out the same purposes as the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, the following description is made with reference to the accompanying drawings, in which:

FIG. 1A is a perspective view of one embodiment of a prosthetic foot device according to the present invention.

FIG. 1B is an exploded view of the prosthetic foot device of FIG. 1A.

FIG. 1C is a side view of the prosthetic foot device of FIGS. 1A and 1B.

FIG. 1D is a top view of a natural foot showing the talocrural (T_{c}) and talocruronavicular (T_{cn}) axes.

FIG. 1E is a top view of the prosthetic foot shown in FIGS. 1A to 1C showing equivalent T_{c} and T_{cn} axes to those shown in FIG. 1D.

FIG. 1F is a side view of a natural foot showing the talocrural (T c) and talocruronavicular (Tcn) axes.

FIG. 1G is a side view of the prosthetic foot shown in FIGS. 1A to 1C showing equivalent T_{c} and T_{cn} axes to those shown in FIG. 1F.

FIG. 1H is a side view of the prosthetic foot device of FIGS. 1A to 1C showing the departure point angle for a heel member.

FIG. 2A is a perspective view of one embodiment of a forefoot device of the present invention.

FIG. 2B is a side view of the forefoot device of FIG. 2A.

FIG. 2C is an end view of the forefoot device of FIG. 2B taken along line 2C-2C.

FIG. 2D is a top view of the forefoot device of FIG. 2B taken along line 2D-2D.

FIG. 3 is a perspective view of a helical coupling implemented in one or more embodiments of the present invention.

FIG. 4 is a perspective view of a T-shaped coupling body according to one embodiment of the present invention.

FIG. 5A is a perspective view of one embodiment of a collar wrap heel mount.

FIG. 5B is a side view of the collar wrap device of FIG. 5A.

FIG. 5C is a top view of the collar wrap device of FIG. 5B taken along line 5C-5C.

FIG. 5D is an end view of the collar wrap device of FIG. 5B taken along line 5D-5D.

FIG. 6A is a perspective view of one embodiment of a heel member according to the present invention.

FIG. 6B is a side view of the heel member of FIG. 6A.

FIG. 6C is an end view of the heel member of FIG. 6B taken along line 6C-6C.

FIG. 6D is a top view of the heel member of FIG. 6B taken along line 6D-6D.

FIG. 7 is a perspective view of accessory, integrated cosmesis shields and mounting hardware.

FIG. 8A is a graph showing the nonlinear moment of resistance in the ankle for an intact person.

FIG. 8B shows a graph of the moment of resistance versus its angle of deflection for a model of an intact ankle.

### DETAILED DESCRIPTION OF THE PREFERRED EMBODIMENTS

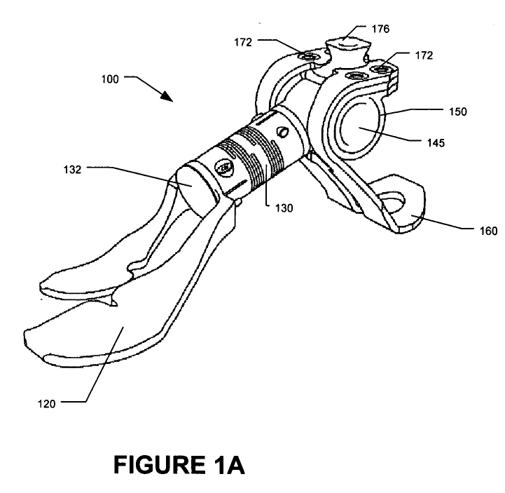

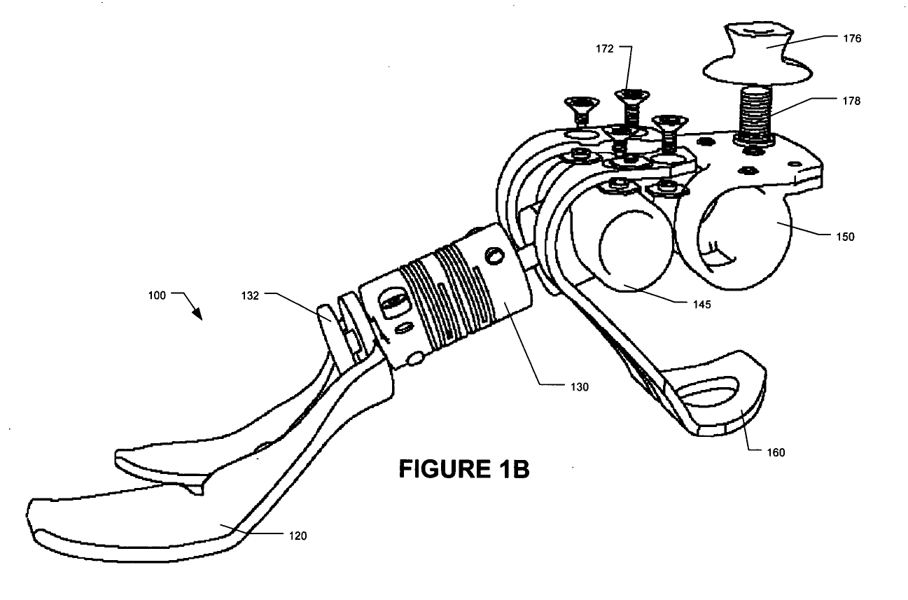

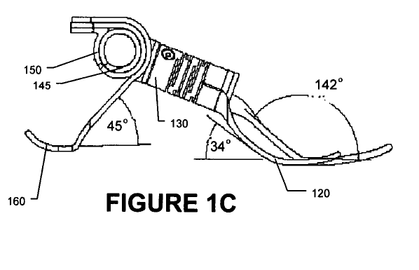

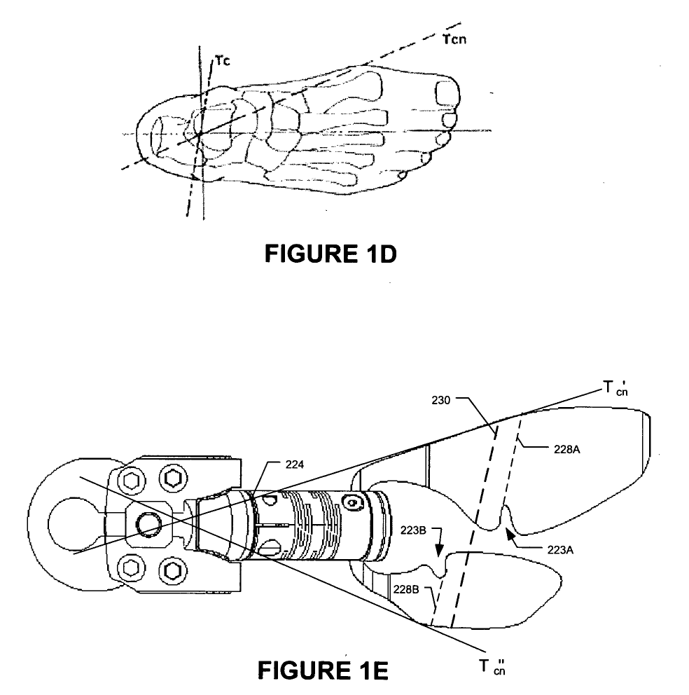

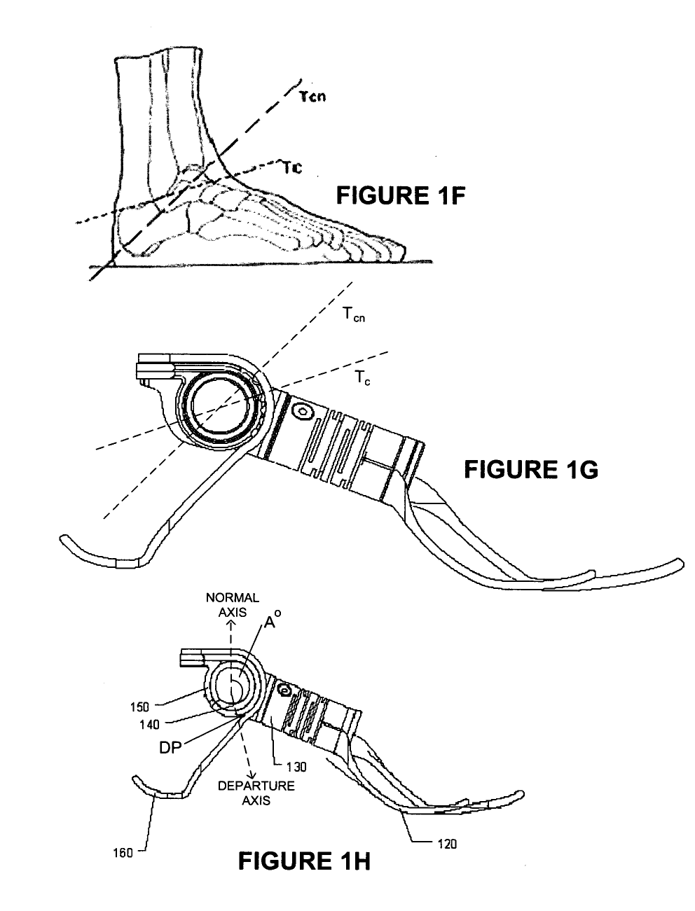

With reference to FIGS. 1A through 1C, there is shown an embodiment 100 of a right side prosthetic foot. (A left side foot is a mirror of the foot 100 and thus will not be discussed.) The foot 100 generally comprises a forefoot portion 120, coupling member 130, and a heel assembly 140, which includes a T-shaped coupling member 145, heel mount device 150, heel member 160, heel assembly fasteners 172, leg mount 176, and leg mount fastener 178. The heel assembly 140 is coupled to a proximal end of the coupling device 130 through the coupling member 145, and the forefoot member 120 is coupled to the distal end of the coupling device 130. In the following sections, the forefoot member 120, coupling device 130, and heel assembly 140 will separately be addressed with regard to their structures and operations.

Coupling

With reference to FIG. 3, along with FIGS. 1A to 1C, rotary coupling 130 will initially be discussed. The rotary coupling 130 has distal and proximal ends for mounting the forefoot 120 and heel assembly 140, respectively. The distal end has a hole 333 for fixedly receiving a mount peg 132 to fix the forefoot 120 to the rotary coupling 130. It has a slit opening 334 that closes in response to bolt 335 being tightened in order to lock mount peg 132 in place and properly secure the forefoot 120. Similarly, the proximal end of rotary coupling 130 has slit opening 337 and lock bolt 338 for receiving and securing the heel assembly 140 in a similar manner.

Rotary coupling 130 may be any suitable device for coupling a forefoot portion to a heel assembly consistent with the principles discussed herein. Preferably, it will constitute a frictionless, low-hysteresis bearing for rotary applications such as a rotary flexure device. Such devices are available from companies such as C Flex Bearing Co, Hexfoil Rotary Flexures, and Heli-cal Products Company, Inc. of Santa Maria, Calif. Ideally, the utilized coupling device will have longitudinal (compressive/expansive) flexibility in the range between 2% and 12%, up to about 6% or so lateral flexibility, but will have rotary (or torsional) flexibility in the range of about 20% to 30%. The depicted rotary coupling 130 is of the double helical flexure arrangement type, as manufactured by the Heli-cal Products Company, Inc. of Santa Maria, CA. The use of such a helical rotary coupling 130 is advantageous for many reasons. Firstly, it is commercially available and relatively simple in that it has no moving parts. Secondly, it allows torsional flexing in both directions about its longitudinal axis with a progressive torsional loading without much (if any) potential backlash on either joined component and replicates the torsional twisting that occurs in a natural foot. Thirdly, the rotary coupling 130 allows some flexing along its longitudinal axis to provide force translation along its length. This allows the prosthetic foot 130 to compact and expand longitudinally without excessive distortion (maximum 6 degrees). Further, this force translation along the length of the prosthetic foot 100 is due to the longitudinal axis of the rotary coupling 130 being oriented through the center of gravity of the foot 100, which results in stability throughout the gait process.

Forefoot Device

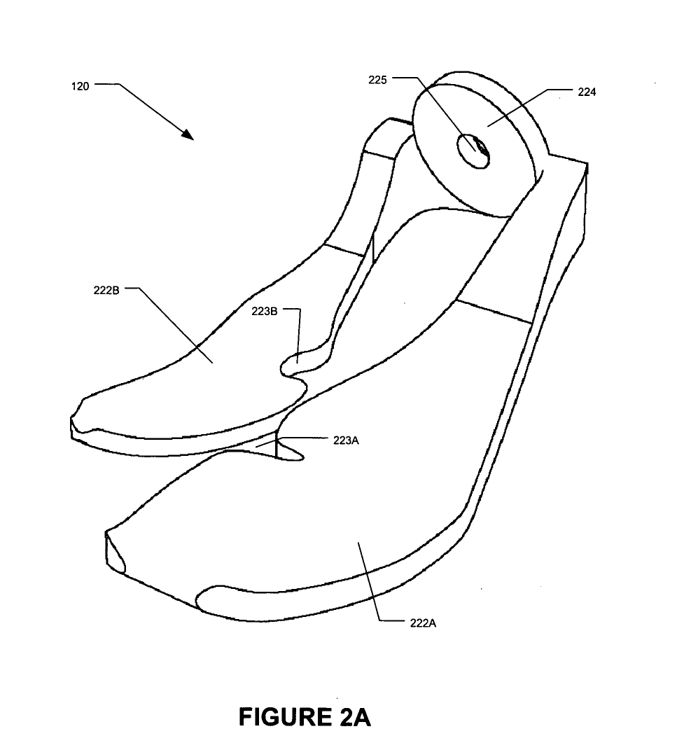

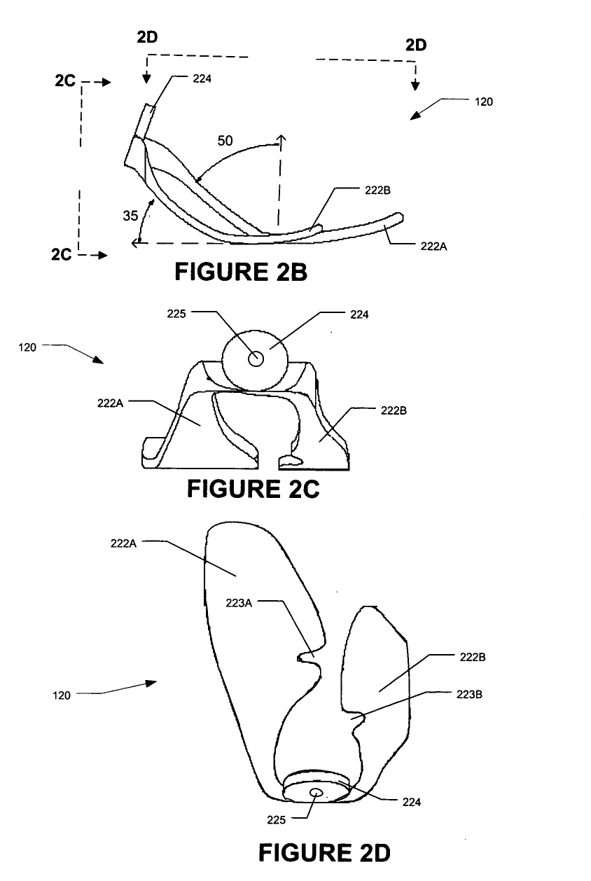

With reference to FIGS. 2A through 2C, one embodiment of a forefoot device 120 is depicted. Forefoot device 120 has a mounting structure 224 with a central hole 225 adapted to receive the mount pin 132 for alignment and connection to the coupling device at its distal end through mount hole 333. The forefoot member 120 also has a relatively long inner foot portion 222A and a shorter outer forefoot portion 222B. The inner portion 222A is also wider than the outer portion 222B and, given that they are of similar thickness, exhibits a lower level of resilience (i.e. a higher level of deformation resistance). When viewed from the side, both of the portions 222A, 222B are slightly concavely curved towards the user's limb.

With reference to FIG. 1E, each of the portions 222A, 222B also have a notch 223A and 223B respectively, which results in a reduction in their cross sectional area and the creation of a weakened line of flexure at regions 228A and 228B. As a result, in use, the mean line of flexure of the forefoot portions 222A, 222B is between the inner and outer flexure regions 228A, 228B, as indicated by the line 230. When the mean line of flexure 230 is compared to the T_{c} axis of the natural foot shown in FIG. 1D, it is evident that the line 230 is substantially parallel to it but forwardly displaced from it. Shifting the mean line of flexure 230 forward of the T_{c} axis in this manner is advantageous as it increases the user's ankle stability while allowing a natural range of motion.

In addition, the location of the two lines of forefoot flexure 228A and 228B closely replicate those provided by the first and fifth metatarsal phalanges of the natural foot, which again increases stability and also improves the adaptation of the user to the prosthetic foot 100. The two forefoot portions 222A, 222B also advantageously distribute the pressure of the body weight on the foot 30 in a manner akin to a natural foot, with the portion 222A handling the majority of the weight loading and the portion 222B primarily acting as a balancing agent.

Also, positioning the flexure lines 228A, 228B at the lowest point of the forefoot portions 222A, 222B, generally ensures that the flexure lines 228A, 228B will make contact with the ground and will not move in response to changes in angle between the forefoot 120 and the ground. This also leads to increased stability and user familiarity.

Indicated at T_{cn}′ and T_{cn}″, FIG. 1E also shows the equivalent T_{cn} axes for the inboard edge of portion 222A and outboard edge of portion 222B, respectively. These axes run, on the one hand, from the mid line of the rotary coupling 130 where it intersects the equivalent center of gravity axis to, on the other hand, the mean line of flexure 230. Because the actual T_{cn} axis for intact anatomy is rotational (and also unbalanced, i.e., approximately 35 degrees inversion to 15 degrees eversion), allowance should be made for equally annular axes, T_{cn}′ and T_{cn}″, e.g., symmetrical at roughly 20 degrees inversion and eversion as designed. When doing this, one should consider the rotational range of movement for the rotary coupling 130. The allowance for the intact anatomy is present with these mirrored axes with regard to the mean flexure axis 230 formed between these edge incident moments. Because of this, there is no conflict of behavior leading to stressful moments on componentry and/or user interface at the stump socket.

The forefoot 120 may be made of any suitable material. In one embodiment, it is made from a unitary carbon composite material as known to persons of ordinary skill in the art.

Heel Assembly

With general reference to FIGS. 1A, 1F to 1H, 4, 5A to 5D, and 6A to 6D, the heel assembly 140 will now be described. As represented in FIGS. 1F and 1G, with the depicted heel assembly 140, more direct translations of the talocrural (T_{c}) and talocruronavicular (T_{cn}) axes are incorporated with the depicted design. The heel assembly 140 is configured so that the intersection of these axes occurs at the center-point of the rotational ankle portion of the heel assembly. This is intended to allow for consistent behavior regardless of the positioning of the forefoot component 120 and rotational coupling 130. With this in mind, the construction and operation of the depicted heel assembly 140 will now be discussed.

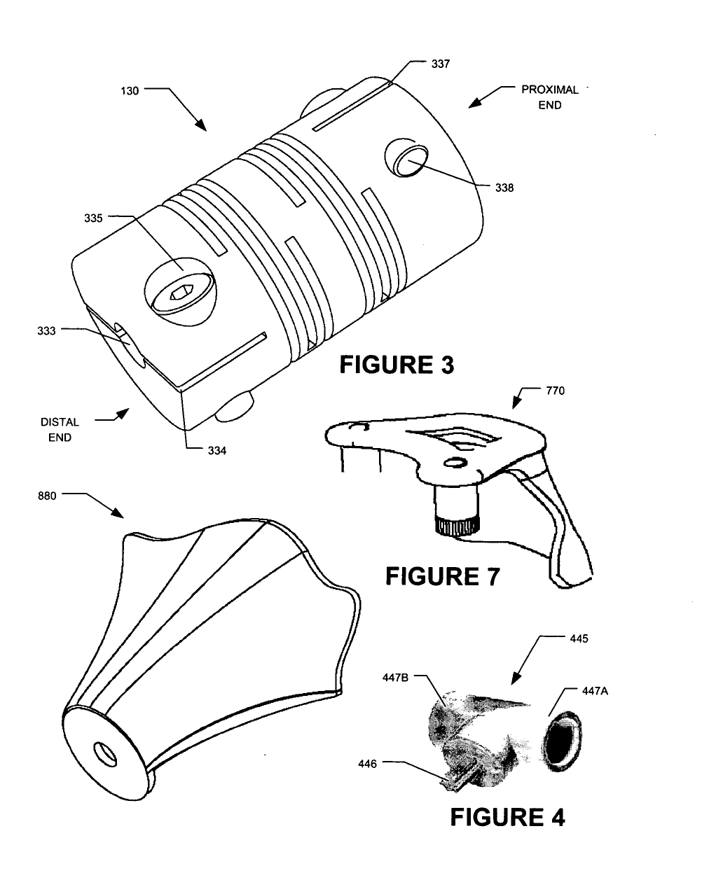

FIGS. 1A and 4 show one embodiment of a coupling member 145, which is a generally T-shaped body having a pair of outwardly facing cylindrical outer surfaces 447A, 447B and a protruding connection insert member 446, which is received by mount opening 337 in the proximal end of rotary coupling 130 in order to fix the rotary coupling 130 to the T-shaped coupling body 145.

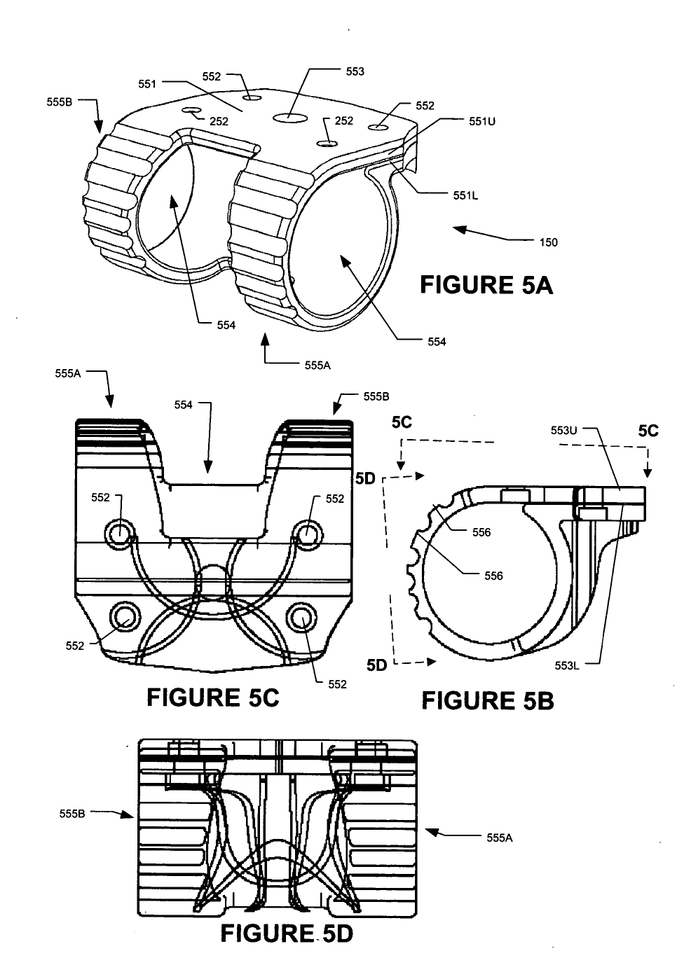

With further reference to FIGS. 5A through 5D, a heel mount device is shown. The heel-mount component 150 serves not only as a locating surface for the internal T-shaped body 145 but also as a locating surface for the heel component 160. Heel mount 150 is positioned around the cylindrical surfaces 447A, 447B of the T-shaped body 145. As seen in the figures, it has a protruding tail portion 551 formed from adjoining upper and lower tail portions 551U and 551L, respectively, along with oppositely-facing outer radial surfaces 555A, 555B (corresponding to the cylindrical surfaces 147A, 147B), which each have a plurality of spaced apart indexing ridges (or bumps) 556. As shown in FIG. 5A through 5D, the heel mount 150 functions akin to a clamping device “clamping together” at upper and lower portions 551U and 551L. The upper and lower portions 551U and 551L have four pairs of openings 552 through which are placed the fasteners (nuts and bolts with countersunk heads) 172 to clamp the heel mount 150 at a desired position about the cylindrical surfaces 447A, 447B of the T-shaped body 145. Tail portion 551 also has a hole 553 for connecting the leg mount 176 to the tail portion 150 with fastener 178.

The clamping provided by the fasteners 172 allows the position of the T-shaped body 145, and thus the rotary coupling 130 and forefoot 120 to be adjusted with respect to the heel assembly 140. The adjustment is made to orient the heel assembly 140, rotary coupling 130 and forefoot 120 in a position most closely replicating that of the user's natural foot and/or shoe being worn. It also allows for the heel member 160 (discussed below) to be adjusted, e.g., for different shoe types or user preferences, relative to the forefoot portion 120 without compromising the operation of the heel assembly 140. It should be recognized that both the T-shaped body 145 and heel mount 150 can be made out of any suitable configuration and from many suitable materials including but not limited to steel, aluminum, titanium and carbon fiber materials.

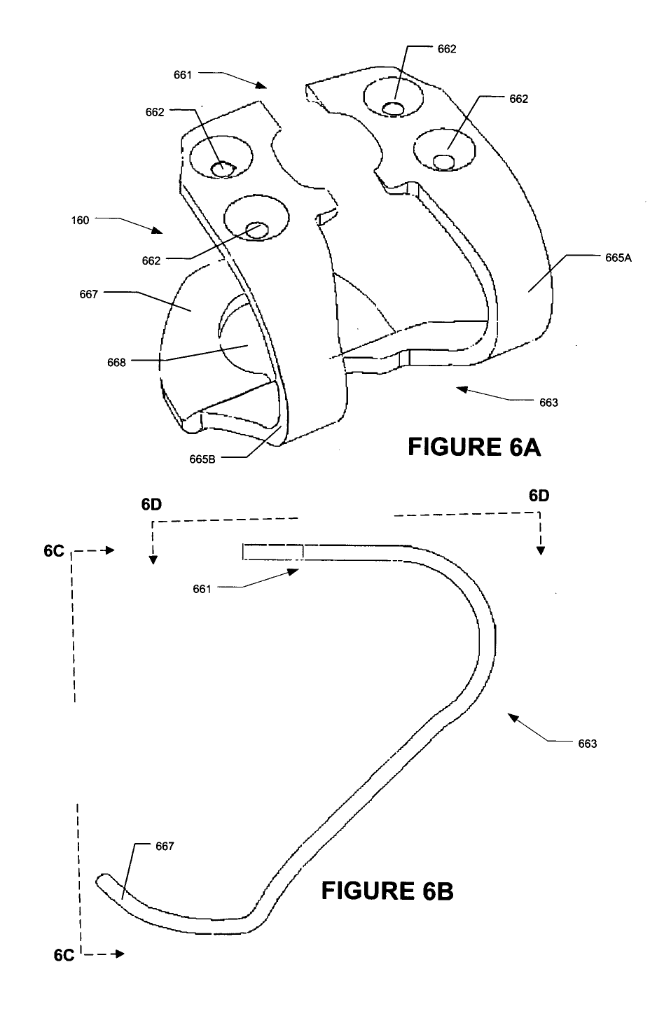

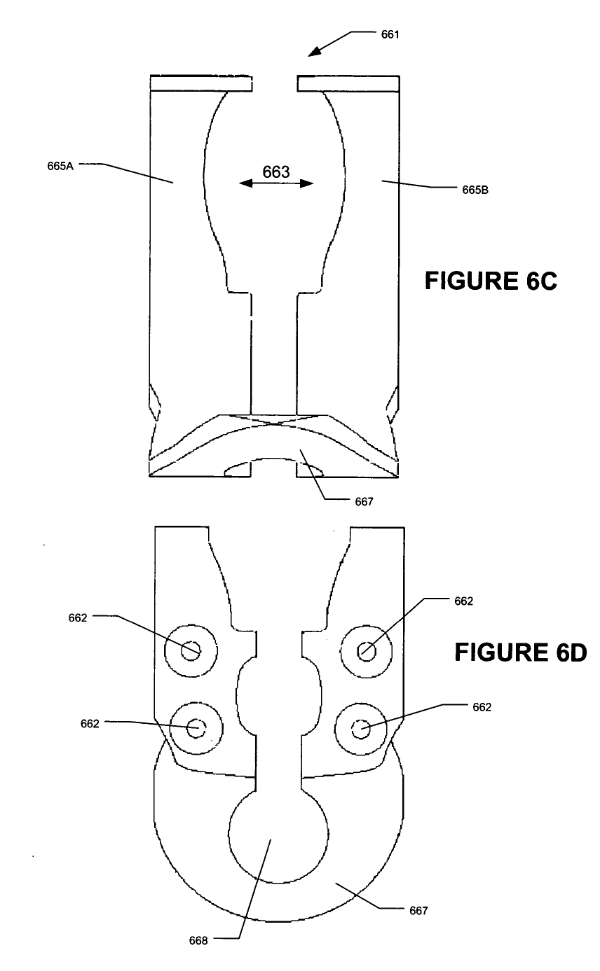

With reference to FIGS. 6A to 6D, the heel member 160 will now be described. Functioning as a rotationally pivoted leaf spring, heel member 160, working in cooperation with heel mount 150, provides a non-linear response to loading similar to intact anatomical musculoskeletal arrangements. It is fixed to the heel mount 150 by the fasteners 172, along with the leg mount and leg mount fastener 176, 178. It is formed as a single component having first and second partially curved support members 665A and 665B coming together into a single U-shaped distal end 667. They each have a proximal end 661 with a flat portion and a curved portion. The flat portions each have a pair of openings 662 through which the fasteners 172 pass to mount the support members 665A, 665B to the mounting clamp 150, and the curved portions are shaped to mount about the radial surfaces 555A and 555B. They also have a vertical, intermediate region 663 that is fairly straight.

The distal end 667 is generally U-shaped with a central hole 668 and, when viewed from the side, has a slightly concave, upward curve towards the user's limb. The shaping of the material of the distal portion 667 into the U-shape around the central hole 668 is done to take advantage of carbon fiber's known improved resistance to failure when subjected to edge loading (i.e. loading about the peripheral edge of the hole 668). This is particularly important as the distal end 667 is the part of the prosthetic foot 100, which makes initial contact with the ground.

The proximal end curved portions of the support members 665A, 665B are curved to substantially replicate the radial outer surface 555A, 555B of the heel mount 150 so that they partially wrap around the radial surfaces 555A, 555B. However, these curved portions are formed slightly out of round and with a non-constant radius of curvature that is approximately equal to that of the surfaces 555A, 555B nearest the fasteners 172 and which increases, relative to the radial surfaces 555A, 555B, as the proximal end 661 progresses towards the intermediate portions 663. As a result of this, the position on the radiused surfaces 555A, 555B where the support members 665A, 665B depart from (or cease to contact) the radiused surfaces 555A, 555B changes depending on the stage in the gait, i.e., on the load exerted on the heel member 160. The variance in this departure point is best explained with reference to FIG. 1H, which shows the prosthetic foot 100 during the part of a normal gait when the foot is subjected to the most weight. This figure depicts an angle of departure, A, between the indicated normal axis and contact axis, which intersects the point on the outer heel mount radial surface 555A/B where a support member 665A/B departs from the surface. This point is called the “departure point” and is identified in FIG. 1H as “DP.” This departure point is at the highest part of the curved surface 555A/B (small departure angle A) when no weight is on the foot and is at the lowest part of the curved surface (large departure angle A) when maximum weight is on the foot 100. With the depicted design, this occurs as pressure is exerted downward onto the foot causing the support members 665A, 665B to in effect wrap around the curved outer heel mount surfaces 555A, 555B. When the foot 100 makes initial impact on the ground and the heel member 160 flexes under the user's load, the angle, A, increases and the departure point DP moves towards the distal end 667 of the heel member 160. As the divergence point DP moves towards the distal end 667, the effective spring-length of the support members 665A, 665B shorten thereby increasing their resistance to deformation.

The changes in effective spring length makes it easier to achieve a desired non linear load response to the load that is placed upon it in order to more closely mimic intact anatomical muscular-skeletal arrangements. This is of course desirable. Put another way, the non linear loading response of the varying effective spring length of the heel member 160 causes initial low resistance with increasing resistance as heel strike progresses. The load response, of course, is also a function of the designed spring characteristics of the heel member 160, itself. For example, if the heel member is made from a carbon fiber laminate composite, factors such as material type, layer density, and ply orientation can be selected, as known to persons of skill in the art, to provide a heel member with desired spring load characteristics. To a certain extent, it can be designed to have a non-linear response, but it has been found that a desired response can be more readily attained by controllably shortening its effective length as it is being depressed (as discussed above) in cooperation with the use of a suitable heel member 160.

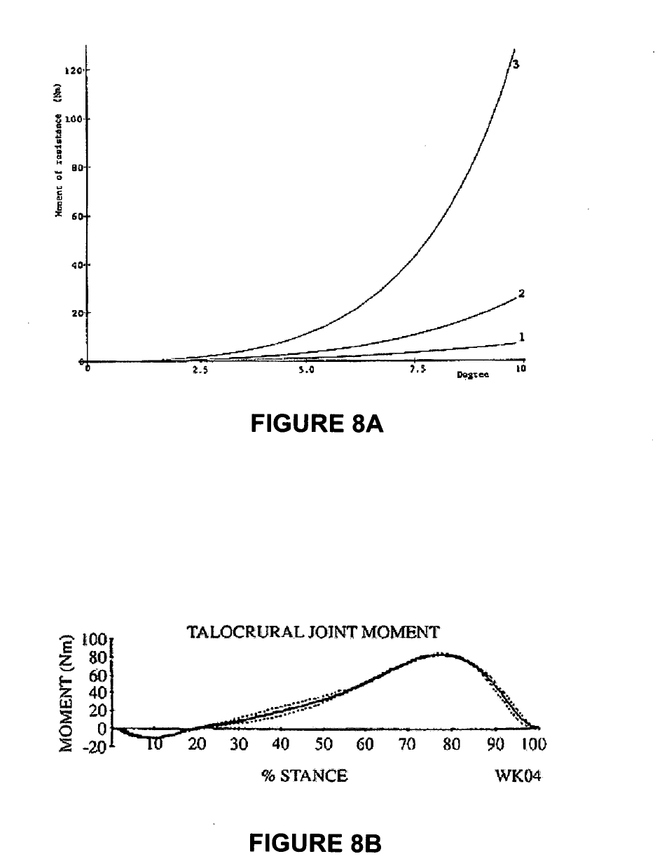

With reference to FIGS. 8A and 8B, in one embodiment, the heel member 160 and heel mount 150 are designed so that the load response of heel assembly 140 corresponds to the nonlinear moment of resistance in the ankle for an intact person, which is depicted in FIG. 8A. One aid to achieving this is through the use of the graph of FIG. 8B, which shows the moment of resistance versus its angle of deflection for a model of an intact ankle, derived by Dr. Mark Pitkin and described in his article entitled, “Mechanical Outcomes of a Rolling Joint Prosthetic.”, American Academy of Orthotists and Prosthetists, Journal of Prosthetics and Orthotics, Vol. 7, No. 4, pp. 114-123 (1995) and its enumerations of the non-linear moment of resistance in the ankle, and the moment of resistance in the ankle versus the angle of deflection. In order to more effectively achieve such a load response, the index ridges 556 are used to achieve known, discrete departure points for the support member parts of the heel member 160 and thereby control the effective heel spring length to be one of a plurality of discrete lengths for mapping the designed load response to the graphs of FIGS. 8A and 8B. Thus, the indexing ridge locations are determined based on this desired load response taking into account the spring characteristics of the utilized heel member 160. That is, the index ridges 556 provide discrete points on the curved surface making it easier to impose the desired load response pursuant to this formula on a piece-wise basis. In addition, the progression of the support members down the outward radial surfaces of the collar wrap 150 encounters the indexing ridges 556, which creates a perceptible progress indication during the phases between heel strike and foot flat, simulating natural proprioception for the amputee. The indexing allows an initial compliance upon heel strike, with progressive resistance compliant to the intact anatomical behavior. This indexing also allows, with a short training period, for relative positioning of the progression of the foot from heel strike to foot flat and helps to avoid “foot slap.” It should also be noted that the amount of distortion of the heel member 160 in response to the user's body weight, and its magnification upon heel strike can be tuned, by the orientation of the carbon fiber within the heel member 160.

Further, FIGS. 1F and 1G show a comparison between the talo-crural axis (T_{c}) and talo-cruronavicular (T_{cn}) axis for an intact foot (FIG. 1F) and equivalent T_{c}′ and T_{cn}′ axes in the prosthetic foot 100. The heel assembly 140 provides a direct translation of the T_{c}′ and T_{cn}′ axes with the intersection of the axes being placed in the center point of the T-shaped coupling member 145, which is about where the distal end 667 of the heel member 160 rotates. This results in constant behavior of the heel assembly 160 regardless of the positioning of the forefoot 120 and the rotary coupling 130 because the heel assembly 140 (and resultant leg orientation to normal) operates independently of the orientation of the forefoot 120 to normal. In other words, the adjustment of the positioning of the rotary coupling 130 and forefoot 120 do not affect the orientation of the components of the heel assembly 140 (and their resultant behaviors with respect to one another and as a whole) during any phase of gait.

Another advantage of the heel assembly 140 is due to the nature of the distal end 667 and separate support members 665A, 665B. More particularly, if one side of the distal end 667 makes contact with the ground before the other then that side of the distal end 667, and only its associated support members 665A or 665B, will distort and create a twisting moment in the coupling member 145. This moment is transferred into the user's limb, thereby providing an indication as to whether the inside or outside of the foot 100 is in contact with the ground.

Heel member 160 may be formed from any suitable material or combinations of materials to provide it with desired spring response characteristics in connection with the design of the other heel assembly components. In the depicted embodiment, heel member 160 is formed from carbon fiber, as an example laminated HSC (high strength carbon) fiber, bonded with either SP Systems' Ampreg 26/prime 20 series resin system for wet lay-up or resin infusion methods. Alternatively, laminated SE 85 pre-preg cloth with OCLV method can be used. As such, it is designed to act akin to a rotationally pivoted leaf spring.

Other

FIGS. 7 and 8 demonstrate how the prosthetic foot 100 can readily accept a forefoot shield 770 and an Achilles heel shield 880. The shields 770, 880 alter the exterior shape of the prosthetic foot 100 to better suit positioning within a conventional shoe for cosmetic considerations.

An overall primary advantage of the embodiment of the prosthetic foot described above is that the three major components (i.e. the heel assembly, coupling and forefoot) individually, and in combination, provide a prosthetic foot, which acts in a manner more closely duplicating that of a natural foot. Further, the major components can all be easily scaled up or down in size to replicate feet of varying sizes. Although the invention has been described with reference to a specific example, it would be appreciated by those skilled in the art that the invention may be embodied in many other forms. For example, it is preferred that the prosthetic foot 100 would be supplied in a kit form with major components of differing sizes, strengths etc that may be assembled by the user in combinations best suited to the activity to be undertaken. This is important as a wide range of market needs exists.

1. A heel assembly for a prosthetic foot, the assembly comprising:
a resilient heel member adapted to be mounted in a prosthetic foot device;
a heel mount adapted for connection in the prosthetic foot, the heel mount having a contact surface for engaging a portion of the heel member to establish in it an effective spring length, wherein the contact surface engages different portions of the heel member for different phases of a gait cycle when the heel member is being loaded thereby effectively shortening the heel member's spring length and providing it with a non-linear loading response as it is being depressed in the gait cycle.

2. The heel assembly of claim 1, wherein the heel mount contact surface has a radial surface, the heel member being adapted to wrap around at least a portion of the radial surface thereby effectively shortening the heel member spring length by an amount corresponding to the amount it wraps around the radial surface.

3. The heel assembly of claim 2, wherein the heel member portion subject to contact with the heel mount contact surface has a radius of curvature that is initially approximately equal to the radius of curvature of the contact surface but increases relative to that of the contact surface as the heel member further compresses in the gait cycle.

4. The heel assembly of claim 3, wherein the heel-mount comprises a substantially q-shaped clamping device.

5. The heel assembly of claim 4, further comprising a rigid coupling body to couple the heel mount to the forefoot, the heel mount being mounted about said rigid coupling body and pivotally adjustable with it in a horizontal axis thereby making the heel assembly pivotally adjustable within the prosthetic foot in a horizontal axis.

6. The heel assembly of claim 4, wherein the heel member includes a pair of spaced apart support members terminating into a substantially U-shaped distal end that makes contact with the ground in a gait cycle.

7. The heel assembly of claim 6, wherein the distal end preferably has an opening therein and is slightly concavely curved towards a proximal end.

8. The heel assembly of claim 1, wherein the heel member is formed from a carbon fiber composite.

9. The heel assembly of claim 1, wherein the contact surface includes indexing ridges that engage the portion of the heel member engaged by the contact surface when the heel member is loaded.

10. A prosthetic foot comprising a heel assembly as set forth in claim 1.

11. A forefoot for a dynamic response prosthetic foot, the forefoot comprising:
a proximal end adapted for mounting within the prosthetic foot;
a distal end concavely curved towards a user's limb, the distal end having a relatively longer and less resilient inner forefoot portion and a relatively shorter and more resilient outer forefoot portion,
the inner forefoot portion having a relatively forward weakened flexure region and the outer forefoot portion having a relatively rearward weakened flexure region, wherein, in use, the mean line of flexure of the forefoot portions is between the outer and inner flexure regions and substantially parallel to, and forwardly displaced from, the T_{c} axis of rotation of an equivalent intact foot.

12. The forefoot of claim 11, wherein the flexure regions are formed by a reduction in cross-sectional area of the forefoot portions proximal to the flexure lines.

13. The forefoot of claim 12, wherein the reduction in cross-sectional area is achieved by removing a notch of material from the forefoot portions adjacent the flexure regions.

14. The forefoot of claim 13, wherein the flexure regions are preferably also positioned to coincide with the region of the forefoot portions proximal to ground contact patch portions.

15. The forefoot of claim 11, wherein the proximal end and distal end are integrally formed from a carbon fiber composite material.

16. A prosthetic foot comprising a forefoot in accordance with the forefoot of claim 11.

17. A prosthetic foot, comprising:
a rotary flexure device having proximal and distal ends;
a forefoot device mounted to the rotary flexure device at its distal end; and
a heel assembly mounted to the rotary flexure device at its proximal end, said heel assembly including: (i) a resilient heel member, and a (ii) a heel mount, the heel mount having a contact surface for engaging a portion of the heel member to establish in it an effective spring length, wherein the contact surface engages different portions of the heel member for different phases of a gait cycle when the heel member is being loaded thereby effectively shortening the heel member's spring length and providing it with a non-linear loading response as it is being depressed in the gait cycle.

18. The prosthetic foot of claim 17, wherein the forefoot includes: (i) a proximal end adapted for mounting to the distal end of the rotary flexure device, and (ii) a distal end concavely curved towards a user's limb, the distal end having a relatively longer and less resilient inner forefoot portion and a relatively shorter and more resilient outer forefoot portion, the inner forefoot portion having a relatively forward weakened flexure region and the outer forefoot portion having a relatively rearward weakened flexure region, wherein, in use, the mean line of flexure of the forefoot portions is between the outer and inner flexure regions and substantially parallel to, and forwardly displaced from, the Tc axis of rotation of an equivalent intact foot.

19. The prosthetic foot device of claim 17, wherein the rotary flexure device is a helical coupling device.
